(19) United States
(12) Patent Application Publication
(10) Pub. No.: US 2024/0378721 A1
(43) Pub. Date: Nov. 14, 2024

(54) METHOD OF NON-INVASIVE MEDICAL TOMOGRAPHIC IMAGING WITH UNCERTAINTY ESTIMATION

(71) Applicants: BATES TOMOGRAPHIC IMAGING LIMITED, St Albans (GB); FRONTWAVE IMAGING, S.L., Barcelona (ES)

(72) Inventor: Oscar Alan BATES, St Albans (GB)

(21) Appl. No.: 18/552,913

(22) PCT Filed: Mar. 31, 2022
(86) PCT No.: PCT/EP2022/058737

§ 371 Date: Sep. 28, 2023

(30) Foreign Application Priority Data
Apr. 1, 2021 (GB) 2104792.3

(51) Int. Cl. G06T 7/00

(52) U.S. Cl. CPC G06T 7/0012; G06T 2207/10072; G06T 2207/20081; G06T 2207/20084

(57) ABSTRACT

Method of non-invasive medical tomographic imaging with uncertainty estimation There is provided a method of generating tomographic medical image data representative of at least a part of a body of a subject. The method comprises the steps of: providing a tomographic observed data set derived from a tomographic measurement of the at least a part of the body of the subject, the tomographic observed data set comprising a plurality of observed data values; providing a generative model comprising one or more latent parameters representative of statistical behaviour of spatial structures of one or more reconstructed medical images; generating, utilising the generative model and from the one or more latent parameters, a spatial model having a plurality of model coefficients; generating, utilising the one or more model coefficients, a predicted tomographic data set comprising a plurality of predicted data values representative of at least one physical parameter; modifying, utilising a gradient-based method, one or more objective functions operable to compare the observed and predicted data values by modifying one or more of the latent parameters to generate updated latent parameters; updating the generative model utilising the updated latent parameters to produce an updated generative model; utilising the updated generative model to generate tomographic medical image data representative of at least a part of the body of the subject for medical analysis.

The present invention relates to an improved method of, and apparatus for, non-invasive imaging of regions of the body using tomographic data. More particularly, the present invention relates to a method for non-invasive imaging of regions of the body using tomographic data with uncertainty determination.

Tomographic imaging comprises a group of methods for producing a single or multi-dimensional image of the internal structures of a structure such as the human body through measurement of directed energy impinging on the structure.

Raw tomographic data by itself does not provide the required image of a structure. Rather, the data needs to be reconstructed to generate image data. Reconstruction using an iterative process is known for tomographic data.

Tomographic images can be post processed to produce additional information for the propose of visualisation. For example, projection images can be generated which show the tomographic information in a single projection, or segmentation algorithms can be used to automatically identify structures within the tomographic images.

Numerous methods for tomographic imaging are known in medical fields. Two commonly used approaches are Ultrasound computed tomography (USCT) and X-ray computed tomography (XCT).

Ultrasound computed tomography comprises directing high frequency acoustic waves (generally at frequencies in excess of 20 kHz) at a structure from one or more orientations and measuring the pressure change produced by the structure over a fixed time interval. Algorithms can then be used to reconstruct physical properties in the volume being imaged. For example, such physical properties includes, compressional sound speed, shear sound speed, or attenuation. A known approach is full-waveform inversion (FWI).

X-ray CT comprises directing X-rays at a structure from one or more orientations and measuring the decrease in intensity along a series of linear paths as a function of X-ray energy, path length, and material linear attenuation coefficient. Algorithms can then be used to reconstruct the distribution of X-ray attenuation in the volume being imaged. A known approach is statistical iterative reconstruction tomography (SIRT).

These tomographic reconstruction processes comprise iterative reconstruction processes. In general, known approaches use a starting estimate (or starting model) for the reconstruction, which is then iteratively modified to generate a final model which is representative of the structure being imaged. Such iterative reconstruction processes generate a predicted data set from the starting model and then iteratively modify the starting model to maximise the similarity (or minimise the difference) between the predicted data set and the observed data set. Often, these processes use the maximum likelihood estimation process.

The use of suitable prior information can assist an iterative reconstruction. Prior information about the general form or structure of the final model can be used to improve the reconstructed image by regularisation. Regularisation is the process of constraining the optimisation in accordance with the prior information so that desirable image characteristics are promoted. Further, the regularisation may comprise a penalty that imposes a cost on an optimisation function for overfitting the function or making the optimal solution unique.

Prior information may comprise one or more prior data sets (also known as “priors”) in two forms: data-space priors which make assumptions about the structure of the measurement data; and image-space priors which make assumptions about the structure of the image.

A known data-space prior is the total variation regularisation which may the used to smooth the reconstructed images.

A known structural prior is the Tikhonov regularisation which may be used to penalise reconstructed images which differ significantly from the prior image. A known approach is to penalise positron emission tomography reconstructions which look incorrect in comparison to a subject-specific MRI image.

However, a known problem with iterative reconstruction is that the accuracy of the final model so generated is often unknown. Iterative processes may get trapped in local, rather than global, minima such that the final model generated may not be accurate. Whilst prior information assists in the process of arriving at an accurate final model, the accuracy of the final model may still not be known with confidence.

This is a particular issue in medical imaging where diagnosis may be made based on the images. In such situations, an indication of the accuracy of the final image is desirable to enable a medical professional to provide a diagnosis based on the images so obtained.

One approach to address this issue is uncertainty estimation. However, uncertainty estimation is uncommon in medical imaging because traditional estimators have excessive computational complexity and because the utility of reconstruction uncertainty is under-appreciated.

Therefore, the known art has drawbacks in terms of accuracy of the estimations provided. The present invention, in embodiments, addresses these technical problems.

According to a first aspect of the present invention there is provided a method of generating tomographic medical image data representative of at least a part of a body of a subject, the method comprising the steps of: a) providing a tomographic observed data set derived from a tomographic measurement of the at least a part of the body of the subject, the tomographic observed data set comprising a plurality of observed data values; b) providing a generative model comprising one or more latent parameters representative of statistical behaviour of spatial structures of one or more reconstructed medical images; c) generating, utilising the generative model and from the one or more latent parameters, a spatial model having a plurality of model coefficients; d) generating, utilising the one or more model coefficients, a predicted tomographic data set comprising a plurality of predicted data values representative of at least one physical parameter; e) modifying, utilising a gradient-based method, one or more objective functions operable to compare the observed and predicted data values by modifying one or more of the latent parameters to generate updated latent parameters; f) updating the generative model utilising the updated latent parameters to produce an updated generative model; g) utilising the updated generative model to generate tomographic medical image data representative of at least a part of the body of the subject for medical analysis.

In one embodiment, step g) further comprises: h) generating one or more reconstructed medical images representative of the at least a part of the body of the subject.

In one embodiment, the or each reconstructed medical image comprises a plurality of image elements representative of values of at least one physical parameter.

In one embodiment, the image elements comprise pixels or voxels.

In one embodiment, the at least one reconstructed medical image comprises a mean tomographic image and an uncertainty image representative of the statistical distribution of the values of at least one physical parameter as a function of image element in the reconstructed medical image.

In one embodiment, a plurality of likely reconstructed medical images is generated, the range of reconstructed medical images being indicative of uncertainty.

In one embodiment, step h) further comprises generating an image representative of the difference between the latent parameters provided in step b) and the updated latent parameters.

In one embodiment, step g) comprises generating the tomographic medical image data from a plurality of model coefficients of a spatial model generated from the updated generative model.

In one embodiment, the model coefficients of the spatial model generated from the updated generative model are quantitative.

In one embodiment, step d) comprises generating the predicted tomographic data set utilising a physics-based model defining a numerical simulation of known physics.

In one embodiment, the physics-based model comprises an acoustic wave equation.

In one embodiment, the physics-based model comprises a machine learning component.

In one embodiment, the latent parameters of the generative model follow a Gaussian distribution.

In one embodiment, the latent parameters of the generative model follow a mean-field Gaussian distribution.

In one embodiment, the generative model is operable to perform unsupervised machine learning.

In one embodiment, the generative model comprises a neural network.

In one embodiment, the generative model comprises a Gaussian model.

In one embodiment, the generative model comprises a mean-field Gaussian model.

In one embodiment, step b) further comprises training the generative model utilising prior information comprising one or more sample data sets.

In one embodiment, the or each sample data set comprises spatial structures representative of one or more reconstructed medical images.

In one embodiment, the or each sample data set comprises one or more ground truth annotations and/or one or more natural images.

In one embodiment, the or each sample data set is generated from observed experimental data and/or by a statistical reconstruction.

In one embodiment, the tomographic observed data set comprises ultrasound image data of the subject acquired from an ultrasound tomographic measurement.

In one embodiment, steps d) to f) are performed using a full waveform inversion method.

In one embodiment, the tomographic observed data set comprises X-ray computed tomography image data of the subject acquired from an X-ray computed tomographic measurement.

In one embodiment, the tomographic observed data set comprises tomographic image data acquired from an imaging modality selected from the group of: positron emission tomography; time of flight tomography; diffraction tomography; electrical impedance tomography; magnetic impedance tomography; and MRI.

In one embodiment, the one or more objective functions comprise a likelihood function arranged to compare the observed and predicted data values and a regularisation function arranged to compare the updated latent parameters with previous latent parameters.

In one embodiment, step e) further comprises minimising/maximising the one or more objective functions using a gradient-based method.

In one embodiment, step e) comprises taking a gradient of the or each objective function with respect to a sample or subset of the latent parameters of the generative model.

In one embodiment, step e) utilises automatic differentiation or adjoint-state methods.

In one embodiment, the model coefficients of the spatial model are representative of the spatial distribution of at least one physical model parameter.

In one embodiment, the model coefficients of the spatial model are quantitative with respect to the at least one physical model parameter.

In one embodiment, the at least one physical parameter comprises sound speed or absorption.

According to a second aspect of the present invention, there is provided a computer system comprising a processing device configured to perform the method of the first aspect.

According to a third aspect of the present invention, there is provided a computer readable medium comprising instructions configured when executed to perform the method of the first aspect.

According to a fourth aspect of the present invention, there is provided a computer system comprising: a processing device, a storage device and a computer readable medium of the third aspect.

Embodiments of the present invention will now be described in detail with reference to the accompanying drawings, in which:

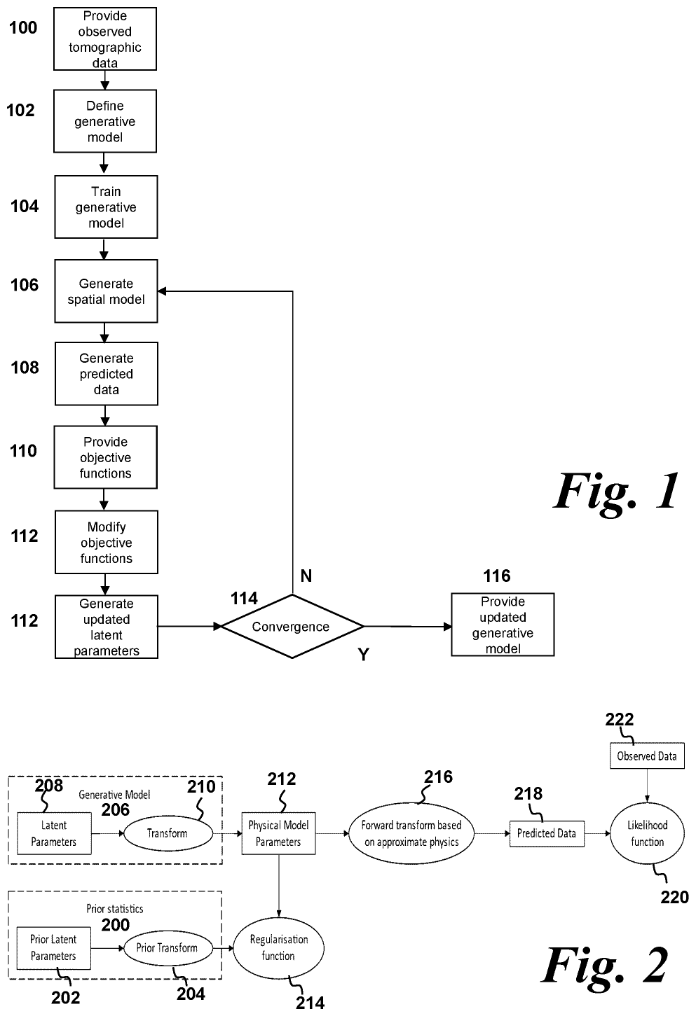

FIG. 1 shows a flow chart according to a first embodiment of the present invention;

FIG. 2 shows a more detailed chart showing features of the first embodiment of the present invention;

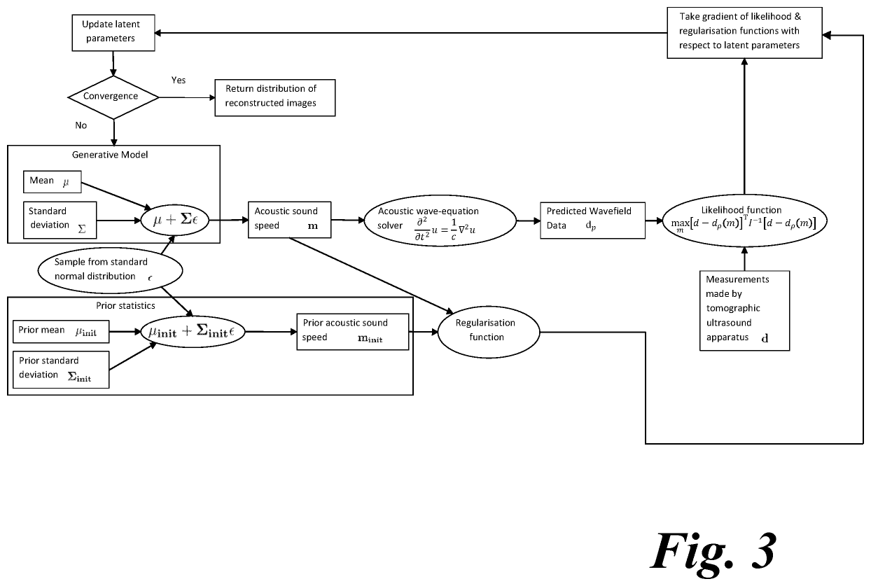

FIG. 3 shows a flow chart of a second embodiment of the present invention utilising ultrasound data and full waveform inversion modelling; and

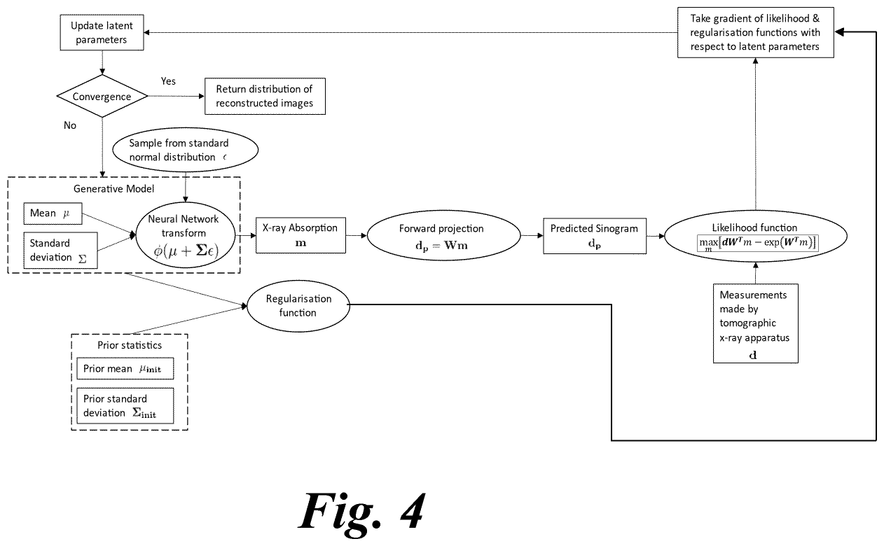

FIG. 4 shows a flow chart of a third embodiment of the present invention utilising X-ray computed tomography data and a statistical iterative reconstruction tomography (SIRT) process.

The present invention, in embodiments, relates to a novel method for tomographic imaging of structures. More particularly, the present invention provides a computationally-efficient and fast process for providing tomographic imaging with uncertainty estimates. FIG. 1 shows a first embodiment of the present invention.

### Step 100: Provide Observed Tomographic Data

At step 100, observed tomographic data is provided. This may be in any suitable form; for example, in non-limiting embodiments it may comprise ultrasound or x-ray data.

The observed tomographic data may be in an appropriate form for the acquisition method used, for example counts per second for XCT, or time domain or the frequency domain pressure measurements for USCT.

If relating to ultrasound acquisition, the observed tomographic data set may be acquired by, for example, recording the waveforms at one or more receivers after emission by one or more sources. The observed data set may then comprise a plurality of waveform traces.

Whilst this step may comprise physical acquisition of the medical data, this is not intended to be limiting and previously-acquired data may simply be provided for use in the following steps. In other words, this step requires a real-world observed tomographic data set to be provided upon which analysis can be performed to facilitate medical imaging of a region of a body of a subject.

The acquired medical data may be provided in any suitable form and may comprise raw data obtained from an acquisition process, or it may contain data which has been pre-processed, formatted or otherwise modified provided it remains representative of medical acquisition tomographic data of a subject.

The method proceeds to step 102.

Step 102: Define generative model

At step 102, a generative model is defined.

A generative model, or generative modelling algorithm, comprises a joint probability distribution of observable variables and unobservable latent variables. Generative models contain a transform describing the relationship between the observable and latent variables. A transform is a sequence of one or more mathematical operations which can be derived mathematically or learned from the data.

A generative model is operable to perform an unsupervised machine learning task to identify patterns and structures in initial input data such that the generative model can be used to generate new data.

A generative model is distinct from a discriminative model in that, when presented with a sample from the latent distribution, a generative model will produce an output which approximates the input data.

In the present invention, the generative model defines the statistical spatial variation of one or more model parameters. For example, in non-limiting embodiments, the model parameter may be acoustic sound speed and the statistical distribution of the sound speed as a function of image element position across a reconstructed image may be encoded in a generative model.

During reconstruction, the generative model so defined is operable to infer a conditional distribution over the latent parameters given an observed tomographic dataset. This approach thus comprises stochastic variational inference.

In non-limiting embodiments, a suitable generative model may be a mean-field Gaussian. In this example, each image element (e.g. pixel, voxel) of the physical model parameters has a unique mean and variance term with no additional covariance terms. The mean (μ) is a vector, the standard deviation (Σ) is a diagonal matrix.

The method proceeds to step 104.

### Step 104: Train Generative Model (Optional)

At step 104 the generative model provided in step 102 is trained using prior information to generate a prior generative model. This is known as an “informative prior” because it is an informed initial estimate of the reconstruction with mean and uncertainty estimates encoded into the prior generative model. This is then utilised in the reconstruction process as described below.

In embodiments, the prior generative model is derived by statistical methods. For example, one or more examples of a sample data set can be used to train the generative model, where the latent parameters and the transform can contain trainable parameters.

The prior information to train the generative model may take a number of different forms.

For example, prior information may be obtained from one or more example reconstructions from different patients using a statistical process such as averaging.

Here, a previous reconstruction could be used, provided this reconstruction is capable of providing distribution information. The reconstruction could be from the same or a different imaging modality, for example a previous ultrasound reconstruction could be used to produce a new ultrasound reconstruction, or an x-ray tomogram could be transformed from relative absorption to sound speed. The x-ray tomogram may not include uncertainty itself; the uncertainty could come from the absorption to sound speed transform.

Alternatively, medical or non-medical natural images could be used which are not specific to the body part of interest. The training process teaches the generative model to recognise correlations in natural objects, meaning it will penalise discontinuities, regions of high curvature or any other forms/correlations not present in natural objects.

A final form of prior information is expert knowledge of how the reconstruction should appear, provided this knowledge leads to uncertainty in the generative model.

Any suitable prior information, including one or more data sets, may be used to train the generative model, provided the training process results in a prior generative model having mean and uncertainty estimates containing structural information about a posterior reconstruction derived from one or more example images or expert knowledge. This is also known as an “informative prior”.

In other words, an informative prior contains estimated information relating to a reconstruction. For example, in the imaging of a head of a subject, the informative prior contains statistical information in the latent space which defines the general structure and format of a representative head of the subject or human anatomy.

Further, the definition of uncertainty within the prior provides an indication of regions of a reconstruction where one or more parameters of a reconstruction (e.g. sound speed) are well defined and regions where significant uncertainty as to the correct values of the one or more parameters exists. In regions of low uncertainty in the prior image, the values of the one or more parameters can be considered to be accurate; in regions of high uncertainty they cannot.

The method proceeds to step 106.

### Step 106: Generate Spatial Model

At step 104, a spatial model comprising one or more model coefficients is generated from the latent parameters using the generative model. In the first iteration, the spatial model is generated from the prior latent parameters.

The generative model is operable to transform the trained latent parameters to spatial parameters to generate the spatial model. The coefficients of the spatial model so generated are representative of the spatial distribution of at least one physical model parameter.

In other words, the generative model comprises a forward transform from a latent distribution which generates a spatial model comprising coefficients which correspond to an expected range of values of at least one physical model parameter across a multi-dimensional space. In a non-limiting embodiment, a physical model parameter may comprise sound speed.

The spatial model may be used to generate one or more reconstructed images if required. Thus the coefficients of the model define a spatial distribution of the one or more physical parameters (e.g. sound speed, absorption) which are then used to define values of one or more image elements (e.g. pixels or voxels) of a reconstructed image. The method proceeds to step 108.

### Step 108: Generate Predicted Data

At step 108, the coefficients of the spatial model generated in step 106 are utilised to generate predicted data using a physics-based model. This may include learned components such as neural networks, but this system will be designed to replicate a numerical simulation of known physics.

As noted, the spatial model comprises, in embodiments, model coefficients which define the spatial distribution of values of at least one physical model parameter. In a non-limiting embodiment, a physical model parameter may comprise sound speed.

The model coefficients are then utilised by a physics-based model to generate predicted data which is comparable to the observed tomographic data obtained in step 100. In non-limiting examples, the physics-based model may comprise the acoustic wave equation which is solved using a numerical method.

This step thus generates predicted tomographic data which is representative of the data which would be acquired if a real-world tomographic measurement was performed on a structure corresponding to the structure of the spatial model generated in step 106.

The method proceeds to step 110.

### Step 110: Provide Objective Functions

The present invention utilises a gradient-based optimisation approach whereby the predicted tomographic data set and observed data set are compared and modified (in step 112) to optimise one or more objective functions (which may involve maximising the similarity or minimising the misfit).

In embodiments, the objective functions comprise a likelihood function and a regularisation function.

The likelihood function is a data-space objective function which is operable match the predicted and observed data by modifying the set of latent parameters of the generative model.

The regularisation function comprises a direct comparison between the updated latent parameters and the prior latent parameters. In a non-limiting case the regularisation is performed in the model image parameter space for FWI and the generative model latent space for XCT. The purpose of the regularisation function is to penalise changes in the latent parameters which are too distant from the distribution of latent parameters in the prior generative model. An alternative embodiment could perform the regularisation with respect to any previous update of the model.

The objective functions measure geometric distance using a divergence metric. In embodiments, the divergence metric may comprise the Kullback-Leibler divergence.

### Step 112: Modify Objective Functions

At step 112, the gradient of the likelihood function and the regularisation function are calculated. This approach comprises stochastic variational inference.

The likelihood function and the regularisation function are modified (in embodiments, minimised/maximised) with respect to the latent parameters on the basis of the gradient. For any useful measure of likelihood the predicted tomographic data set will move towards the observed tomographic data set, regularised by the regularisation function to penalise latent parameters which diverge significantly from those previously defined.

In embodiments, the iterative optimisation utilises gradient-based methods where a gradient or higher order derivative is calculated with respect to the parameters of the generative model.

One approach is to utilise gradient descent, which comprises a dynamic forward propagation followed by a backward propagation. However, alternatives may be used. For example, gradient unrolling may be used which replaces the backward propagation with a neural network formulation.

The method proceeds to step 114.

### Step 114: Generate Updated Latent Parameters

Based on the results of the update involving modification (e.g. minimisation/maximisation) in step 112, at step 114 updated latent parameters are generated. The method proceeds to step 116.

### Step 116: Convergence Criteria Met?

At step 116 it is determined whether convergence criteria have been met. For example, the method may be deemed to have reached convergence when the difference between the data sets reaches a threshold percentage or other value. If the criteria as set out above have been met, then the method proceeds to step 118 and the final spatial model is generated.

Convergence may be defined as a function having a predetermined metric, with convergence achieved once this metric reaches, exceeds or falls below a predefined threshold. In embodiments, non-limiting examples of metrics may include the combined likelihood and regularisation terms, the mean value of a region of the uncertainty distribution, or a prespecified number of iterations.

If the criteria have not been met, then the method proceeds back to repeat steps 106 to 112 as discussed above where an updated spatial model having updated model coefficients is generated from the updated latent parameters in step 112.

### Step 118: Provide Updated Generative Model

At step 118, it is deemed that the method has converged on an output that is deemed to be sufficiently accurate to generate a final updated generative model comprising the updated latent parameters.

The final updated generative model may be used for medical analysis. This may involve the direct interpretation of a recovered final spatial model generated from the generative model. Alternatively or additionally, the final spatial model could be utilised to generate a reconstructed image.

Additionally or alternatively this may involve the process of generating one or more reconstructed images from the generative model utilising the updated latent parameters. This may involve sampling from the latent parameter distribution and transforming the sample to produce an image.

The or each reconstructed medical image comprises a plurality of image elements (pixels or voxels) representative of the anatomy or structure being imaged. The or each reconstructed image could have representative values of at least one physical parameter such as sound speed.

In other words, the reconstructed medical image comprises a spatial distribution of image features which may be representative of tissues or parts of the body of the subject.

At least one reconstructed image may be generated, and the generative model is used to determine a range of reconstructions or values for each image element (e.g. pixel or voxel) which are indicative of uncertainty.

The uncertainty estimate, in simple terms, enables areas of the reconstructed image which may be less accurate to be identified. This may assist in further image processing or in subsequent diagnosis.

With regard to image processing, segmentation of the image based on relative uncertainty may be used to identify and segment features in the image such as image artefacts caused by the experimental acquisition errors.

With regard to the application to a subsequent diagnosis, a medical practitioner or other analyst may identify a potential feature of interest in the reconstructed image.

Conventionally, there would be significant doubt as to whether this feature was a real physical entity (e.g. a tumour or other growth) or an artefact of the image processing or reconstruction.

However, by providing an uncertainty estimate representative of the statistical distribution of the values of at least one physical parameter as a function of the image element (e.g. pixel or voxel) in the reconstructed medical image, a measure of uncertainty can be assigned to this feature. A feature of interest which has a relatively low uncertainty could then reasonably be considered to be real, whereas a feature of interest having a high uncertainty may warrant caution or further investigation. Areas of high and low accuracy may optionally be segmented to focus on regions of high accuracy, for example.

FIG. 2 shows the mathematical computation features of an embodiment of the present invention which apply in the tomographic imaging process described in steps 100 to 116 of FIG. 1.

As shown, the prior generative model 200 is shown in FIG. 2 represented as “prior statistics”. The prior generative model 200 comprises prior latent parameters 202 which are trained in step 104 and a prior transform 204 from latent space to image space.

The generative model 206 is updated in each iteration of the iterative optimisation process of steps 106 to 114 and comprises updatable latent parameters 208 and a transform 210 to transform the latent parameters to produce a spatial model having model coefficients representative of one or more physical model parameters 212.

These, together with the output of prior transform 204 are input to the regularisation function 214 which is optimised in step 112. This approach ensures that the regularisation is dependent upon the measure of uncertainty, with large updates from relatively certain features penalised and large updates from relatively uncertain features encouraged.

The forward transform 216 is operable to generate predicted tomographic data 218 from the image parameters of the physical model defined in step 108. The predicted tomographic data 218 is input into the likelihood function 220 along with the observed tomographic data 222 and these elements are compared to generate an update to the latent parameters 208.

In embodiments, the regularisation and likelihood functions could be observed in different spaces (for example the latent parameter space, the physics model parameter space, or the dataspace). The reconstruction process according to the present invention requires a latent space, a transform, and a physics model. In an alternative embodiment, a physics model could be used in reverse to predict some physical model parameters, which was then used to train the generative model.

In the method of the present invention, informative prior information is required due to the significant role of the regularising term as a constraint. This is necessary when complex physics is included, such as electrical impedance tomography which uses a highly non-linear physics model which fails to produce meaningful tomographic reconstructions without strict structural constraints.

The regularisation is also beneficial for artefact reduction, such as suppressing streaking artefacts in X-ray CT.

The inventor has realised, for the first time, that the use of a generative model with a process based on stochastic variational inference can, when applied to medical imaging, have significant advantages over the known art.

The method of the present invention utilises stochastic variational inference (SVI) to train a generative model to produce a distribution of reconstructions based on tomographic data. By applying this to medical imaging in the present invention, this process results in a low-cost estimate of the reconstruction uncertainty. The estimation of mean and uncertainty information using a generative model produces a distribution of reconstructions which are suitable given the observed data in one or more iterations.

The present invention has numerous advantages over known arrangements.

First, common regularisation terms (for example Total Variation and Tikhonov) cannot make assumptions about associated uncertainty. However, the use of uncertainty within the prior generative model can effectively penalise large updates to the reconstruction where the prior is most certain and enables large updates to the reconstruction where the prior is least certain. Therefore, the prior generative model encourages reconstructions with the correct structural appearance.

Secondly, the present invention operates in two distinct phases. As a first stage the prior generative model may be trained with respect to the general statistical structural form of the body part of the subject to be imaged. Then, the latent parameters of the prior generative model are updated to produce a generative model (the posterior) which is subject-specific because it is trained with respect to actual subject-specific data (the observed dataset).

Third, many diseases present with anomalous physical behaviour, for example they can be more dense or stiff that healthy tissue. The posterior generative model defines the tomographic reconstruction as a probability distribution; thus, the generative model quantifies the uncertainty with respect to the physical parameter being measured. Uncertainty will improve quantitative diagnostics by providing a range of plausible values rather than presenting a single value, which will enable clinicians to make more nuanced assessments using quantitative imaging modalities.

In this way, diagnostics can be developed which consider both prior and posterior information in a probabilistic way. Probabilistically significant changes between the posterior and the prior can be identified and highlighted, using probabilistic significance tests that have uncertainty estimates from both the posterior and the prior.

Probabilistically significant anomalous physical properties can also be identified, for example unusually dense/stiff tissues, which can identify the disease status of the anomaly. The traditional generative model lacks uncertainty information about the subject specific posterior, meaning anomalies can only be identified based on prior uncertainty and that the physical property cannot be analysed probabilistically.

Finally, the parameterisation of the reconstruction using a generative model enables iterative reconstruction using stochastic variational inference (SVI). SVI can produce estimators which have no additional computational cost compared to standard reconstruction. Therefore, SVI is a computationally fast approach to estimate uncertainty, and does not impose significant extra requirements on computer memory.

The use of SVI means the uncertainty associated with the patient data can be trained into the generative model with minimal additional computational cost compared to alternative methods such as “data to model” reconstructions by Bayesian neural networks, reconstruction ensembling, Markov Chain Monte Carlo or Variational Bayesian methods.

The current invention also keeps the traditional relationship between the data and the reconstruction by delegating the reconstruction of the tomographic image to a traditional reconstruction algorithm or physics-based method. This means large paired datasets aren't required to train the generative model, and therefore the generative model with example data in various formats. Bayesian Neural Networks are expressive, but they are also expensive in 3D and need lots of training data.

Embodiments of the invention will now be described with reference to medical technology applications.

### EXAMPLE 1: ULTRASOUND AND FULL WAVEFORM INVERSION (FWI)

An example of full waveform inversion (FWI) in combination with ultrasonic measurement will now be described. FWI is a known method for analysing data. FWI is able to produce models of physical properties such as acoustic sound speed in the observed region that have high fidelity and that are well resolved spatially. FWI seeks to extract the acoustic properties of the imaged region of the subject from the recorded observed data set. A detailed sound speed estimate can be produced using an accurate model with variations on the scale of the ultrasonic wavelength.

The FWI technique involves generating a two or three dimensional model to represent the observed portion of a subject's head or body region and attempting to modify the properties, coefficients or parameters of the model to generate predicted data that matches the experimentally obtained ultrasonic observed data.

FWI is a tomographic reconstruction algorithm which uses a numerical simulator in the forward transform. In this example, the physical model parameter is the acoustic sound speed (discretised into voxels), and the forward transform is the isotropic acoustic wave equation with constant density. Other instances could include variable density, absorption, or the elastic wave-equations. Examples of a numerical simulator could be finite difference, finite element, pseudo-spectral or spectral element, others are possible. Crucially the numerical simulator models the “full” wave propagation of the wavefield, unlike ray-based methods for example.

First, a hemispherical array of 1024 single-element ultrasound transducers are used to enclose a sample, such as a human head, for example. An ultrasound wavefield is generated using one of the single-element transducers, the wavefield propagates through, and is modulated by the sample. The resulting modulated wavefield is collected by all 1024 single-element transducers in receive mode. These measurements constitute a received ultrasound wavefield.

Measurements made in accordance with this process generate a observed tomographic data set.

Prior latent parameters are generated. Structural prior information is specified in the latent space. The latent space prior so defined takes a statistical form. The example uses mean-field gaussian variational inference.

The generative model is a mean-field gaussian, which means each voxel of the physical model parameters has a unique mean and variance term with no additional covariance terms. The mean (μ) is a vector, the standard deviation (Σ) is a diagonal matrix. The sound speed coefficients (m) are found through the following transform

$$m = μ + \sum ϵ$$

Where the standard deviation matrix is multiplied by a vector (ϵ) containing samples from the standard normal distribution (ϵᵢ˜ [symbol] (0,1)).

The acoustic wave equation is solved using a numerical method:

$$ς ( {x}_{s} ) = {L ( m )}^{T} {u}_{p} ( m ) \\ ς ( {x}_{s} ) = \frac{{\partial }^{2}}{\partial {t}^{2}} {u}_{p} ( m , x ) - \frac{1}{m ( x )} {∇}^{2} {u}_{p} ( m , x )$$

The output of the numerical solver is the predicted data (dₚ). The predicted data is compared to the observed/observed data (d) using a likelihood function.

The KL-divergence is used to measure distance between the true posterior (p(m| [symbol] ) and the predicted posterior (q(m)), where [symbol] is the set of observed and predicted data. The KL-divergence can be arranged in the form:

$$1 ) \begin{matrix}{q}^{*} ( m ) = {max}_{q ( m )} {\int }_{m} - q ( m ) ln [ p ( 𝒟 \vert m ) ] - q ( m ) ln [ \frac{p ( m )}{q ( m )} ] dm \\ {max}_{q ( m )} {\int }_{m} - q ( m ) ln [ p ( 𝒟 \vert m ) ] - q ( m ) [ ln [ p ( m ) ] - ln [ q ( m ) ] ] dm\end{matrix}$$

The first term in Equation 1 is the likelihood distribution (p( [symbol] |m)) and the second term is the regularisation term. The likelihood distribution is chosen to be gaussian (other distributions are possible). The gaussian likelihood distribution leads directly to the L2-norm likelihood function, and we solve for each latent parameter individually:

$$2 a ) {μ}^{*} = {{min}_{μ} [ d - {d}_{p} ( m ) ]}^{T} {I}^{- 1} [ d - {d}_{p} ( m ) ]$$

$$2 b ) {Σ}^{*} = {{min}_{Σ} [ d - {d}_{p} ( m ) ]}^{T} {I}^{- 1} [ d - {d}_{p} ( m ) ]$$

Note again that predicted tomographic data (dₚ) is found by Equation 0 and the observed tomographic data (d) is measured experimentally. Also, the reparameterisation of the physical model parameters means that the model is a function of the mean and standard deviation m(μ, Σ)=μ+Σϵ. Note that the second term in Equation 1 is the regularisation term, and it is another KL-divergence between the prior (p(m)) and the predicted posterior (q(m)).

Recall that the prior could be informative, in this case the mean and variance of the prior p(m) could be pretrained using one or more human heads (in the case of one head this could be a representation of the subject's actual head but this would still include an estimate of the variance). The regularisation terms take the form:

$$3 a ) {μ}^{*} = {{min}_{μ} [ m - {m}_{init} ]}^{T} {I}^{- 1} [ m - {m}_{init} ]$$

$$3 b ) {Σ}^{*} = {{min}_{Σ} [ m - {m}_{init} ]}^{T} {I}^{- 1} [ m - {m}_{init} ]$$

Therefore equation 2 minimises the difference between the current model parameters (m) and the prior model parameters (mᵢₙᵢₜ) with respect to the latent parameters of the mean-field gaussian.

Equations 2 and 3 form a single cost function. Equations 2a & 2b seek the set of latent parameters which best match the predicted data to the observed data and Equations 3a & 3b penalise changes to the latent parameters which are too distant from the prior distribution. In traditional FWI, the likelihood is optimised by gradient descent. In this example, the sound speed parameters are reparameterised using a generative model, meaning stochastic variational inference is necessary to calculate gradients with respect to the latent parameters of the generative model. The stochastic variational inference gradients are:

$$4 ) \begin{matrix}\frac{\partial }{\partial μ} p ( 𝒟 \vert g ( λ ) ) = \frac{1}{2} \frac{\partial }{\partial m} {[ d - {d}_{p} ( m ) ]}^{T} [ d - {d}_{p} ( m ) ] \frac{\partial }{\partial μ} ( μ + {\sum }^{T} ϵ ) \\ = {[ \frac{\partial {u}_{p} ( m )}{\partial m} ]}^{T} {X}_{r}^{T} [ d - {d}_{p} ( m ) ]\end{matrix}$$

$$5 ) \begin{matrix}\frac{\partial }{\partial Σ} p ( 𝒟 \vert g ( λ ) ) = \frac{1}{2} \frac{\partial }{\partial m} {[ d - {d}_{p} ( m ) ]}^{T} [ d - {d}_{p} ( m ) ] \frac{\partial }{\partial Σ} ( μ + {\sum }^{T} ϵ ) \\ = {{ϵ}^{T} [ \frac{\partial {u}_{p} ( m )}{\partial m} ]}^{T} {X}_{r}^{T} [ d - {d}_{p} ( m ) ]\end{matrix}$$

In Equations 4 and 5, the predicted wavefield (uₚ(m)) and predicted tomographic data (dₚ) are related by the receiver positions matrix (Xᵣ) such that dₚ(m)=Xᵣuₚ(m).

Note the similarity between the Equation 4 and Equation 5. The derivative of the wavefield with respect to the model parameters is expensive to calculate, so an Adjoint State Method is used. The same adjoint state method can be used to solve Equations 4 & 5. The adjoint state method starts by taking the derivative of the forward problem in Equation 0 with respect to the physical model parameters:

$$6 ) \begin{matrix}ς ( {x}_{s} ) = {L ( m )}^{T} {u}_{p} ( m ) \\ 0 = {L ( m )}^{T} \frac{\partial }{\partial m} {u}_{p} ( m ) + {{u}_{p} ( m )}^{T} \frac{\partial }{\partial m} L ( m ) \\ \frac{\partial {{u}_{pred} ( m )}^{T}}{\partial m} = {{u}_{pred} ( m )}^{T} {\frac{\partial L ( m )}{\partial m} [ {L ( m )}^{- 1} ]}^{T}\end{matrix}$$

where L(m) represents the acoustic wave equation operator, which generates an ultrasound wavefield uₚ(m) from the source displacements [symbol] (xₛ). For the next step, note that the inverse acoustic wave equation operator [L(m)⁻¹]^{T} and the data residual δ(xᵣ)=d−dₚ(m) (at the receivers) represents a new “adjoint” wave equation assuming the operator is invertible:

$$\begin{matrix}{u}_{adj} ( m ) = {[ {L ( m )}^{- 1} ]}^{T} δ ( {x}_{r} ) \\ = {[ {L ( m )}^{- 1} ]}^{T} [ d - {d}_{p} ( m ) ( μ + {Σ}^{T} ϵ ) ]\end{matrix}$$

Hence, applying Equation 6 to Equation 5 shows that the adjoint state method is a correlation between the forward propagating wavefield from the sources, and a backward propagating “adjoint” wavefield from the data residual at the receivers.

$$7 a ) \begin{matrix}\frac{\partial }{\partial μ} p ( 𝒟 \vert g ( λ ) ) = \frac{1}{2} \frac{\partial }{\partial m} {[ d - {d}_{p} ( m ) ]}^{T} [ d - {d}_{p} ( m ) ] \frac{\partial }{\partial μ} ( μ + {Σ}^{T} ϵ ) \\ = {[ \frac{\partial {u}_{p} ( m )}{\partial m} ]}^{T} {X}_{r}^{T} [ d - {d}_{p} ( m ) ]\end{matrix}$$

$$7 b ) \begin{matrix}\frac{\partial }{\partial Σ} p ( ( D ) \vert g ( λ ) ) = {ϵ}^{T} {{u}_{p} ( m )}^{T} {\frac{\partial L ( m )}{\partial m} [ {L ( m )}^{- 1} ]}^{T} [ d - {d}_{p} ( m ) ( μ + {Σ}^{T} ϵ ) ] \\ = {ϵ}^{T} {{u}_{p} ( m )}^{T} \frac{\partial L ( m )}{\partial m} {u}_{adj} ( m )\end{matrix}$$

As the term in Equation 6 is duplicated in Equations 4 & 5, the same adjoint state method solution can be used for the derivative of the predicted wavefield in Equation 7a & 7b. Therefore, this uncertainty estimator is far easier to compute than the Hessian approach or Monte Carlo-based solutions. It also makes no assumptions beyond those of made when FWI is solved using the traditional variational method; the mean estimator and the traditional FWI reconstruction are asymptotically identical.

The regularising term is also optimised by gradient descent. The derivatives with respect to the latent parameters are:

$$8 a ) \frac{\partial }{\partial μ} 𝕂𝕃 [ q ( m ) ‖ p ( m ) ] = [ m - {m}_{init} ]$$

$$8 b ) \frac{\partial }{\partial \sum } 𝕂𝕃 [ q ( m ) ‖ p ( m ) ] = [ m - {m}_{init} ] ϵ$$

Combining the regularisation gradients in Equations 8 with the respective likelihood gradients in Equations 7 yields the latent parameter updates for the iterative process. The iterative process ends once convergence is achieved. Convergence is observed using a function as a metric, and convergence is achieved once this metric fall below a certain value. Examples of a metric include the combined likelihood and regularisation terms, the mean value of a region of the uncertainty image, or a prespecified number of iterations.

### EXAMPLE 2: APPLICATION TO X-RAY CT IMAGING

There are many approaches to iterative reconstruction in X-ray computed tomography, although the physics model often assumes a straight-ray X-ray beam with no scattering.

Using these assumptions, the forward model is a linear combination of a weight (or kernel) matrix (W), which describes the contribution of each voxel (m) to the projection data (dₚ). Each voxel is an estimate of the x-ray absorption of the sample observed in Hounsfield units [HU], this the weight matrix tells us the how much each pixel contributes of the total absorption along the ray-path.

Mathematically:

$$9 ) {d}_{p} = Wm$$

The latent parameters are assumed to follow to mean-field gaussian distribution (μ, Σ). However, a neural network (ϕ(⋅)) is also used as part of the generative model which parameterises the physical model parameters (X-ray absorption). Therefore, the generative model is m=ϕ(μ+Σϵ) as described in equation 11 below.

First, observed tomographic data is obtained using a tomographic x-ray apparatus, for example an x-ray source which produces X-rays. The X-rays then travel through a sample subject to a digital radiography photodiode detector.

The measurements comprise the number of photons recorded at each pixel of the photodiode, when all the pixels are combined this forms a projection image measurement (d). The measurements are assumed to have a Gaussian distribution, so the measurements and predicted sinogram are compared using a Gaussian likelihood:

$$10 a ) {μ}^{*} = {{max}_{μ} [ d - {d}_{p} ( m ) ]}^{T} {I}^{- 1} [ d - {d}_{p} ( m ) ]$$

$$10 b ) {Σ}^{*} = {{max}_{Σ} [ d - {d}_{p} ( m ) ]}^{T} {I}^{- 1} [ d - {d}_{p} ( m ) ]$$

where the reparameterisation of the physical model parameters means that the model is a neural network, parameterised by a mean-field gaussian latent space:

$$m = ϕ ( μ + Σϵ )$$

In this case the regularisation function is easiest to apply in the latent-space because we can continue to use the L2-norm regularisation

$$12 a ) {μ}^{*} = {{min}_{μ} [ g - {g}_{init} ]}^{T} {I}^{- 1} [ g - {g}_{init} ]$$

$$12 b ) {Σ}^{*} = {{min}_{Σ} [ g - {g}_{init} ]}^{T} {I}^{- 1} [ g - {g}_{init} ]$$

where the change of variables equation is

$$13 ) g = μ + Σϵ$$

The optimal absorption image is optimised by stochastic variational inference. Taking the derivative of Equation 10 with respect to the latent parameters:

$$14 a ) \frac{\partial }{\partial μ} ℒ = {W}^{T} [ d - Wm ] \frac{\partial ϕ ( g )}{\partial g}$$

$$14 b ) \frac{\partial }{\partial Σ} ℒ = {W}^{T} [ d - Wm ] \frac{\partial ϕ ( g )}{\partial g} ϵ$$

In this case, the derivative of the neural network is found by back-propagation, this is a vector which is inserted into the Equation 14a and 14b. The derivative of regularisation term is similar to Equation 8.

This example can thus be used to generate a reconstructed image based on the X-ray CT scan.

The above examples are not intended to be limiting and the skilled person would be readily aware of the suitable environments in which data could be gathered for imaging and analysis purposes as set out in the present disclosure.

For example, whilst the above examples have been illustrated with respect to ultrasound and X-ray CT imaging modalities, other imaging modalities may also be used. For example, tomographic image data could acquired from one or more of: positron emission tomography; time of flight tomography; diffraction tomography; electrical impedance tomography; magnetic impedance tomography; and MRI.

Further, whilst the present invention has been described with reference to an iterative process, this need not be the case and a single iteration or non-iterative process may be used to arrive at an updated generative model which may be utilised for medical analysis.

In aspects, the embodiments described herein relate to a method. However, the embodiments described herein are equally applicable as an instruction set for a computer for carrying out said method or as a suitably programmed computer.

The methods described herein are, in use, executed on a suitable computer system or device running one or more computer programs formed in software and/or hardware and operable to execute the above method. A suitable computer system will generally comprise hardware and an operating system.

The term ‘computer program’ is taken to mean any of (but not necessarily limited to) an application program, middleware, an operating system, firmware or device drivers or any other medium supporting executable program code.

The term ‘hardware’ may be taken to mean any one or more of the collection of physical elements that constitutes a computer system/device such as, but not limited to, a processor, memory device, communication ports, input/output devices. The term ‘firmware’ may be taken to mean any persistent memory and the program code/data stored within it, such as but not limited to, an embedded system. The term ‘operating system’ may taken to mean the one or more pieces, often a collection, of software that manages computer hardware and provides common services for computer programs.

The methods described herein may be embodied in one or more pieces of software and/or hardware. The software is preferably held or otherwise encoded upon a memory device such as, but not limited to, any one or more of, a hard disk drive, RAM, ROM, solid state memory or other suitable memory device or component configured to software. The methods may be realised by executing/running the software. Additionally or alternatively, the methods may be hardware encoded.

The method encoded in software or hardware is preferably executed using one or more processors. The memory and/or hardware and/or processors are preferably comprised as, at least part of, one or more servers and/or other suitable computing systems.

1. A method of generating tomographic medical image data representative of at least a part of a body of a subject, the method intended for non-invasive imaging of regions of the body, and the method comprising the steps of:
a) providing a tomographic observed data set derived from a tomographic measurement of the at least a part of the body of the subject, the tomographic observed data set comprising a plurality of observed data values;
b) providing a generative model comprising one or more latent parameters representative of statistical behaviour of spatial structures of one or more reconstructed medical images;
c) generating, utilising the generative model and from the one or more latent parameters, a spatial model having a plurality of model coefficients;
d) generating, utilising the one or more model coefficients, a predicted tomographic data set comprising a plurality of predicted data values representative of at least one physical parameter;
e) modifying, utilising a gradient-based method, one or more objective functions operable to compare the observed and predicted data values by modifying one or more of the latent parameters to generate updated latent parameters;
f) updating the generative model utilising the updated latent parameters to produce an updated generative model;
g) utilising the updated generative model to generate tomographic medical image data representative of at least a part of the body of the subject for medical analysis; and
wherein the coefficients of the spatial model define a spatial distribution of the at least one physical parameter which are then used to define values of one or more image elements of a reconstructed image.

2. A method according to claim 1, wherein step g) further comprises:
h) generating one or more reconstructed medical images representative of the at least a part of the body of the subject.

3. A method according to claim 2, wherein the or each reconstructed medical image comprises a plurality of image elements representative of values of at least one physical parameter.

4. (canceled)

5. A method according to claim 3, wherein the at least one reconstructed medical image comprises a mean tomographic image and an uncertainty image representative of the statistical distribution of the values of at least one physical parameter as a function of image element in the reconstructed medical image.

6. A method according to claim 2, wherein a plurality of likely reconstructed medical images is generated, the range of reconstructed medical images being indicative of uncertainty.

7. A method according to claim 2, wherein step h) further comprises generating an image representative of the difference between the latent parameters provided in step b) and the updated latent parameters.

8. A method according to claim 1, wherein step g) comprises generating the tomographic medical image data from a plurality of model coefficients of a spatial model generated from the updated generative model.

9. (canceled)

10. A method according to claim 1, wherein step d) comprises generating the predicted tomographic data set utilising a physics-based model defining a numerical simulation of known physics.

11. (canceled)

12. A method according to claim 10, wherein the physics-based model comprises a machine learning component.

13. A method according to claim 1, wherein the latent parameters of the generative model follow a Gaussian distribution.

14. A method according to claim 13, wherein the latent parameters of the generative model follow a mean-field Gaussian distribution.

15. A method according to claim 1, wherein the generative model is operable to perform unsupervised machine learning.

16. A method according to claim 1, wherein the generative model comprises a neural network.

17. (canceled)

18. (canceled)

19. A method according to claim 1, wherein step b) further comprises training the generative model utilising prior information comprising one or more sample data sets.

20. A method according to claim 19, wherein the or each sample data set comprises spatial structures representative of one or more reconstructed medical images.

21. A method according to claim 19, wherein the or each sample data set comprises one or more ground truth annotations and/or one or more natural images.

22. (canceled)

23. A method according to claim 1, wherein tomographic observed data set comprises ultrasound image data of the subject acquired from an ultrasound tomographic measurement.

24. (canceled)

25. A method according to claim 1, wherein the tomographic observed data set comprises X-ray computed tomography image data of the subject acquired from an X-ray computed tomographic measurement.

26. (canceled)

27. A method according to claim 1, wherein the one or more objective functions comprise a likelihood function arranged to compare the observed and predicted data values and a regularisation function arranged to compare the updated latent parameters with previous latent parameters.

28. (canceled)

29. (canceled)

30. A method according to claim 1, wherein step e) utilises automatic differentiation or adjoint-state methods.

31. A method according to claim 1, wherein the model coefficients of the spatial model are representative of the spatial distribution of at least one physical model parameter.

32. (canceled)

33. (canceled)

34. A computer system comprising a processing device configured to perform the method of claim 1.

35. A computer readable medium comprising instructions configured when executed to perform the method of claim 1.

36. (canceled)
